(19) United States
(12) Patent Application Publication
(10) Pub. No.: US 2018/0099124 A1
(43) Pub. Date: Apr. 12, 2018

(54) SYSTEM AND METHOD FOR CROSSING A NATIVE HEART VALVE WITH A GUIDEWIRE

(71) Applicant: Medtronic Vascular, Inc., Santa Rosa, CA (US)

(72) Inventors: Gerard McLoughlin, Blackrock (IE); Christopher Murphy, Galway (IE)

(21) Appl. No.: 15/286,933

(22) Filed: Oct. 6, 2016

(51) Int. Cl. A61M 25/09

(52) U.S. Cl. CPC A61M 25/09041; A61M 2205/332; A61M 2230/005; A61M 2230/06

(57) ABSTRACT

A system for crossing a heart valve with a guidewire includes an advancement motor and a controller. The controller controls when the advancement motor advances and retracts the guidewire. The controller is coupled to an electrocardiogram device and determines the systolic and diastolic phase of the heart from information/data from the electrocardiogram device. The guidewire advances or retracts based on the controller's determination of the systolic or diastolic phase corresponding with the heart valve being in an open configuration. The system may include a catheter including a lumen through which the guidewire is disposed. The system may further include a sensor. The sensor is in communication with the controller, and the controller will stop advancement of the guidewire if the controller determines the guidewire has not advanced between open leaflets of the heart valve based upon information/data from the sensor.

### FIELD OF THE INVENTION

The present invention relates to a system and method for crossing a native heart valve with a guidewire. More particularly, the present invention relates to systems and methods for crossing a native heart valve of a beating heart with a guidewire based on an electrocardiogram.

### BACKGROUND

Heart valves may sometimes be damaged by disease or by aging, resulting in problems with the proper functioning of the native heart valve. Heart valve replacement may be a viable surgical procedure for certain patients suffering from valve dysfunctions. However, attendant with traditional open surgery significant patient trauma and discomfort may occur, extensive recuperation times may be required, and life threatening complications may occur due the invasive nature of the surgery and the necessity for stoppage of the heart during such a surgery.

To address these concerns, efforts have been made to perform cardiac valve replacements using minimally invasive techniques. In certain methods, laparoscopic instruments may be employed to make small openings through the patient's ribs to provide transapical access to the heart. While considerable effort has been devoted to such techniques, wide spread acceptance has been limited by the clinician's ability to access only certain regions of the heart using laparoscopic techniques.

Still other efforts have been focused upon percutaneous transcatheter (or transluminal) delivery and implantation of replacement cardiac valves to solve the problems presented by traditional open surgery and minimally invasive surgical methods utilizing laparoscopic instruments. Typically, a guiding catheter is first inserted through an incision and into a femoral artery, for instance, of a patient. For example, the Seldinger technique may be utilized for percutaneously introducing the guiding catheter. A guidewire may be introduced through the guiding catheter and maneuvered/advanced through the vasculature to a treatment site, such as the diseased native heart valve. In such methods, the guidewire must be positioned to cross the diseased native heart valve of a beating heart, in order to enable at least a distal portion of a subsequently introduced heart valve delivery system to be desirably disposed within the heart valve during implantation of the replacement cardiac valve.

In a known method of crossing a native heart valve of a beating heart, repeated advancement (also known as pecking) of a guidewire against the anatomy surrounding the native heart valve enables a clinician to feel his/her way into the correct position for crossing the heart valve. As the native heart valve closes and opens, such as occurs with an aortic valve during diastole and systole of the beating heart, it is important for the clinician to time the peck to coincide with the opening of the native heart valve. In some cases, crossing of the native heart valve with the guidewire in this manner may be difficult and undesirably time consuming, and/or may require prolonged fluoroscopy, which in certain instances may adversely affect the patient's health. In addition, repeated pecking with a distal end of the guidewire may in certain instances cause damage to the anatomy.

Moreover in severe cases when the native heart valve cannot be crossed with a guidewire, more invasive options of valve replacement may need to be considered. However, even when crossing of the native heart valve with a guidewire is successful using the practice described above, one or more complications may occur including ventricular arrhythmias, coronary occlusion by dissection, cardiac perforation, heart block, emboli, and/or thrombi. Accordingly, there exists a need for an improved device and method for crossing a diseased native heart valve of a beating heart that reduces the time required to cross the native heart valve and reduces the possibility of one or more of the aforementioned deficiencies of known apparatus and methods.

### SUMMARY OF THE INVENTION

Embodiments hereof relate to a system for crossing a heart valve of a beating heart with a guidewire. The system includes an advancement motor and a controller. The advancement motor is connected to the guidewire and advances and retracts the guidewire. The controller is coupled to an electrocardiogram device and determines the systolic phase and the diastolic phase of a patient's heart from information/data received from the electrocardiogram device. The advancement motor advances the guidewire based on the controller's determination of the systolic phase or the diastolic phase corresponding with the heart valve being in an open configuration.

Embodiments hereof also relate to a method for crossing a native heart valve of a beating heart with a guidewire. The guidewire is coupled to an advancement motor that advances and retracts the guidewire. The guidewire is advanced to a first side of the native heart valve. The heart is monitored with an electrocardiogram device to detect systolic and diastolic phases of the heart. The electrocardiogram device is coupled to a controller, which is coupled to the advancement motor. When the controller determines from information/data received from the electrocardiogram device that the heart is in the systolic phase or the diastolic phase that corresponds with the heart valve being in an open configuration, the guidewire is advanced from the first side of the native heart valve towards a second side of the native heart valve automatically using the advancement motor. If the guidewire does not advance to the second side of the native heart valve, the guidewire is retracted.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is side view of a system for driving or pecking a guidewire based on an electrocardiogram in accordance with an embodiment hereof.

FIG. 2 is a block diagram of an embodiment of the system of FIG. 1 showing the inputs and outputs to a controller of the system.

FIG. 3 is a depiction of a handle assembly of the system of FIG. 1 in accordance with an embodiment hereof.

FIG. 4 is a side view of a distal end of a catheter being used with or forming a component of the system of FIG. 1 in accordance with an embodiment hereof.

FIG. 5 is a side view of a system for driving or pecking a guidewire based on an electrocardiogram in accordance with another embodiment hereof with a sensor disposed on a guidewire in accordance with an embodiment hereof.

FIGS. 6-15 are simplified illustrations of a method of crossing a native heart valve of a beating heart utilizing a system for driving or pecking a guidewire based on an electrocardiogram in accordance with an embodiment hereof.

### DETAILED DESCRIPTION

Specific embodiments of the present invention are now described with reference to the figures, wherein like reference numbers indicate identical or functionally similar elements. The terms “distal” and “proximal”, when used in the following description to refer to a guidewire, catheter, and/or other system component hereof are with respect to a position or direction relative to the treating clinician. Thus, “distal” and “distally” refer to positions distant from, or in a direction away from the treating clinician, and the terms “proximal” and “proximally” refer to positions near, or in a direction toward the clinician. The terms “distal” and “proximal”, when used in the following description to refer to a native vessel or native valve are used with reference to the direction of blood flow from the heart. Thus, “distal” and “distally” refer to positions in a downstream direction with respect to the direction of blood flow and the terms “proximal” and “proximally” refer to positions in an upstream direction with respect to the direction of blood flow.

The following detailed description is merely exemplary in nature and is not intended to limit the invention or the application and uses of the invention. Furthermore, there is no intention to be bound by any expressed or implied theory presented in the preceding technical field, background, brief summary, or the following detailed description.

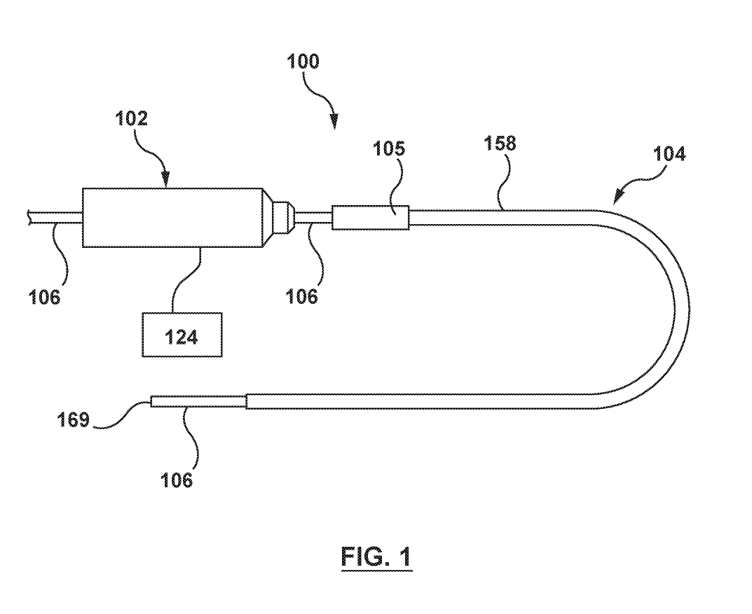

FIGS. 1-4 illustrate a system 100 for driving or pecking a guidewire based on an electrocardiogram (ECG) in accordance with an embodiment hereof. The system 100, as shown in FIG. 1, includes a guidewire 106 having a handle assembly 102, and a catheter 104 (such as, but not limited to a guide catheter). In embodiments hereof, any standard guidewire may be used as the guidewire 106, and such guidewire may be adapted to be connected to the handle assembly 102 in accordance herewith.

The system 100 is configured for crossing a native heart valve, as described in greater detail below. In accordance with methods described herein, once the guidewire 106 has crossed the native heart valve, the handle assembly 102 is configured to be removed to allow the catheter 104, such as a guide catheter, to be removed and swapped out with another medical device, such as a prosthetic heart valve delivery catheter, that may then be introduced over the indwelling guidewire 106. The system 100 is merely an exemplary embodiment of a system for driving or pecking a medical device, such as a guidewire, based on an electrocardiogram, and modifications may be made to the embodiments described herein without departing from the spirit and scope of the present invention. For instance, the guidewire 106 having the handle assembly 102, and the catheter 104, as shown and described below are by way of example and not limitation as variations thereof are contemplated to be within the spirit and scope of the present invention as would be readily apparent to one of ordinary skill in the art. As well based upon application, components of the system 100 may assume different forms and construction, and/or may be modified or replaced with differing structures and/or mechanisms, without departing from the spirit and scope of the present invention. Therefore, the following detailed description is not meant to be limiting.

The guidewire 106 of the system 100 is configured to be advanced through a patient's vasculature to a native heart valve (not shown in FIGS. 1-4). The system 100 is further configured to automatically control the advancement/retraction of the guidewire 106 as the guidewire 106 attempts to cross a native heart valve of a beating heart when the native heart valve is in an open configuration, as described in greater detail below. Stated another way, the system 100 uses an electrocardiogram to synchronize/time the advancement/retraction of the guidewire 106 with the open configuration of the native heart valve for crossing the native heart valve.

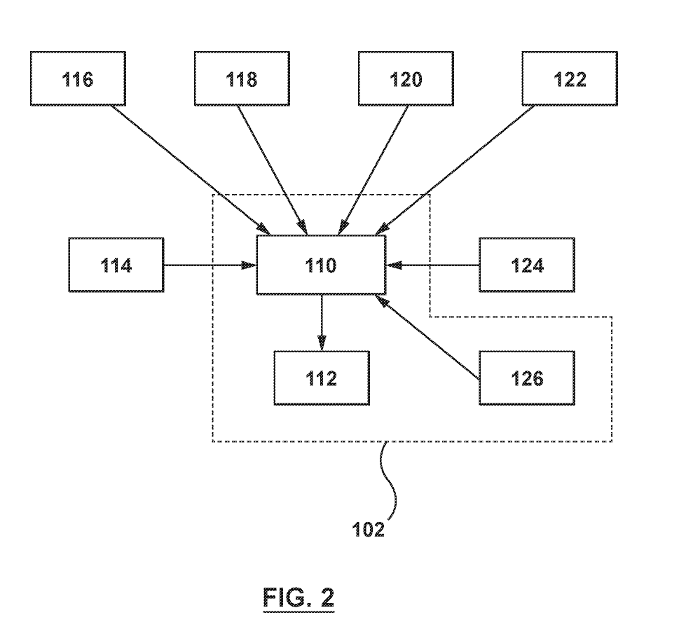

FIG. 2 illustrates an embodiment of the system 100 in a block diagram. The handle assembly 102 (depicted as dashed lines) of the system 100 includes a controller 110 and an advancement motor 112. The controller 110 may be any electronic control unit suitable for providing the system 100 with the various functionalities described herein. In an embodiment, the controller 110 includes a processor (CPU) configured for running software suitable for determining the systolic phase and the diastolic phase of a beating heart from analyzing information/data from an electrocardiogram device 124, and suitable for calculating and controlling the various components of the system 100. The controller 110 is configured for communication with inter alia the advancement motor 112, an actuator mechanism 126, the electrocardiogram device 124, and the sensor 114, if present. As well, the controller 110 is operable to accept user selected control settings, such as from one or more of a peck speed adjustment 116, a peck frequency adjustment 118, a peck depth adjustment 120, and/or a systolic/diastolic delay adjustment 122. The controller 110 is further configured to generate and communicate (transmit) control signals based on the electrocardiogram device 124, a user selected control setting or settings (such as a user selection made via the peck speed adjustment 116, the peck frequency adjustment 118, the peck depth adjustment 120, and/or the systolic/diastolic delay adjustment 122), a user selected setting for the actuator mechanism 126, and the sensor 114. Communication to and from the controller 110 may be made by any suitable communication link, for example, and not by way of limitation, a wireless connection, cable connectors, or any other means capable of allowing communication to occur between components of the system 100 and suitable for the purposes described herein.

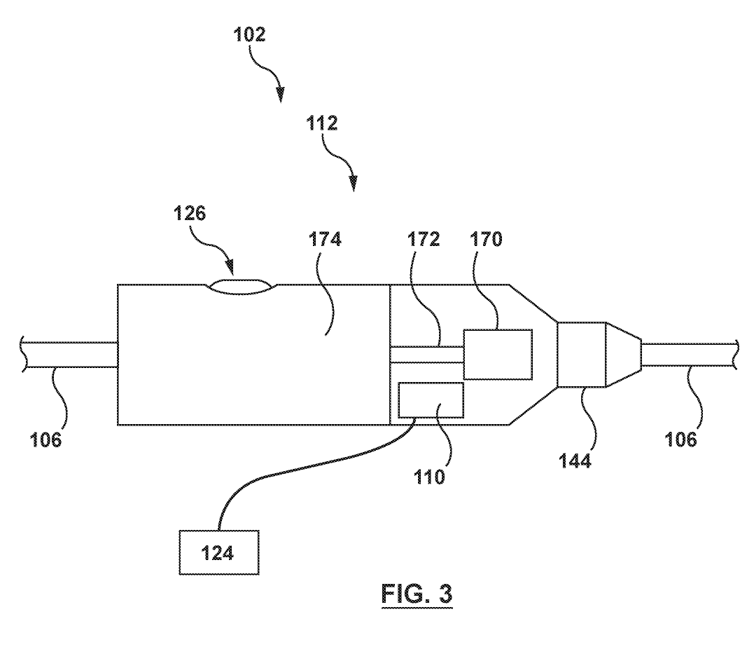

While the controller 110 is shown in FIGS. 2 and 3 as included within the handle assembly 102, this is not meant to limit the design and other configurations may be utilized including, but not limited to the controller 110 as a stand-alone or dedicated unit, a general-purpose system, or other configurations suitable for the purposes described herein.

In an embodiment shown in FIG. 3, the handle assembly 102 includes the controller 110, the advancement motor 112, the actuator mechanism 126, and a clamping device 144. In the embodiment shown in FIG. 3, at least a portion of the actuator mechanism 126 extends beyond an outer surface of the handle assembly 102 for interfacing by a user. The handle assembly 102 is configured for convenient handling and grasping by a user. The handle assembly 102 may be formed, by way of example and not limitation, of polyvinylchloride (PVC), high-density polyethylene (HDPE), polyethylene terephthalate (PET), also acrylonitrile butadiene styrene (ABS), or any other material suitable for purposes embodiments hereof. While the handle assembly 102 is shown in FIG. 3 with a cylindrical shape, this is not meant to limit the design, and other shapes and sizes may be utilized.

The controller 110, as shown in FIG. 2, is in communication with the advancement motor 112 as previously described. The controller 110 is configured to communicate with and to control the actions of the advancement motor 112 as described in greater detail below.

The controller 110 is in communication with the peck speed adjustment 116 as previously described and shown in FIG. 2. The peck speed adjustment 116 is configured to adjust the millimeters per second (mm/sec) of advancement or retraction of the guidewire 106 based upon the peck speed adjustment 116 setting defined by the user. Stated another way, the peck speed adjustment 116 adjusts how fast the guidewire 106 is advanced or retracted.

The controller 110 is in communication with the peck frequency adjustment 118 as previously described and shown in FIG. 2. The peck frequency adjustment 118 is configured to adjust the pecks per minute (ppm) of pecks (advancement and retraction) of the guidewire 106 based upon the peck frequency adjustment 118 setting defined by the user. Stated another way, the peck frequency adjustment 118 adjusts how many times (cycles) per minute the guidewire 106 is advanced or retracted

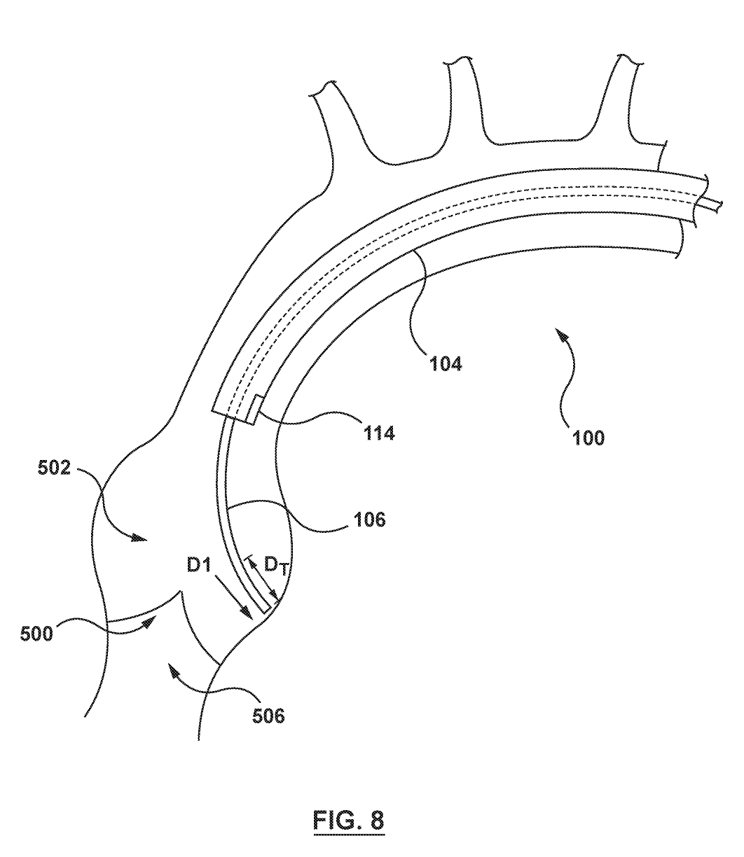

The controller 110 is in communication with the peck depth adjustment 120 as previously described and shown in FIG. 2. The peck depth adjustment 120 is configured to adjust a generally longitudinal travel distance D_{T} (FIG. 8) in millimeters (mm) with respect to the handle assembly 102 upon each advancement peck of the guidewire 106 based upon the peck depth adjustment 120 setting defined by the user. Stated another way, the peck depth adjustment 120 adjusts how far (mm) the guidewire 106 is advanced or retracted with each peck.

The controller 110 is in communication with the systolic/diastolic delay adjustment 122 as previously described and shown in FIG. 2. The systolic/diastolic delay adjustment 122 is configured to adjust a timing of the peck in milliseconds (msec) with respect to the predicted open configuration of the native heart valve based upon the systolic/diastolic delay adjustment 122 setting defined by the user. More specifically, the systolic/diastolic delay adjustment 122 advances/delays timing of the peck with respect to a determined systolic/diastolic peak to account for any lead/lag in the electrocardiogram device 124 information/data with respect to the actual open configuration of the native heart valve. Stated another way, the systolic/diastolic delay adjustment 122 advances/delays (msec) the timing of the peck of the guidewire 106 to fine-tune the synchronization of the peck with the open configuration of the native heart valve. In embodiments hereof, prediction of an open configuration may be based on a number of beats in a previous time period. This time period could be adjustable by a clinician to allow for an irregular heart rhythm e.g. prediction based on sequence in previous 5 seconds or previous 20 seconds depending on the patient.

The controller 110 is in communication with the actuator mechanism 126, as shown in FIG. 2. Activation of the advancement motor 112 is enabled or disabled by the controller 110 based upon a specific state of the actuator mechanism 126, as described in greater detail below.

In an embodiment, the controller 110 is in communication with the sensor 114, as previously described and shown in FIG. 2. The sensor 114 is a force-sensor configured to generate and communicate a continuous signal to the controller 110 of a resistance force (pressure) on a tip 169 of the guidewire 106, as described in greater detail below. In embodiments hereof, the sensor 114 may be a piezoelectric or a piezoresistive sensor. Increased resistance force (pressure) on the tip 169 of the guidewire 106, as determined by controller 110, is indicative of the tip 169 of the guidewire 106 advancing into tissue rather than between open leaflets of the heart valve. In an embodiment, the controller 110 disables movement of the guidewire 106 upon determination of the resistance force (pressure) that is indicative of the tip 169 of the guidewire 106 advancing into tissue. Stated another way, if the resistance force (pressure) on the tip 169 of the guidewire 106 is determined to be of a sufficient force by the controller 110 to indicate that the guidewire 106 has advanced into tissue and has not crossed between open leaflets of the native heart valve, the controller 110 stops movement of the guidewire 106.

In another embodiment, the controller 110 commands the advancement motor 112 to retract the guidewire 106 upon determination that the resistance force (pressure) is indicative of the tip 169 of the guidewire 106 advancing into tissue and not crossing between open leaflets of the native heart valve.

The controller 110 is in communication with the electrocardiogram device 124 and is configured to determine the systolic and the diastolic phases of the patient's heart, and uses information/data received therefrom to predict when the native heart valve will be in the open configuration. Communication to and from the controller 110 and the electrocardiogram device 124 may be made by any suitable communication link, for example, and not by way of limitation, a wireless connection, cable connectors, or any other means capable of allowing communication to occur between components of the system 100 and suitable for the purposes described herein.

The peck speed adjustment 116, the peck frequency adjustment 118, the peck depth adjustment 120, and the systolic/diastolic delay adjustment 122, as shown in FIG. 2, generally referred to as the controls, may be any electronic control suitable for providing the system 100 with the various functionalities described herein. The controls may be digital or analog in nature and are user configurable. The controls may be disposed within the handle assembly 102 (FIG. 3) or they may be external to the handle assembly 102. The controls communicate with the controller 110 as previously described.

The electrocardiogram device 124, as shown in FIG. 2, may be any electrocardiogram device suitable for providing the system 100 with the various functionalities described herein. The electrocardiogram device 124 is configured to detect and communicate electrical activity of a heart over a period of time to the controller 110. Stated another way, the electrocardiogram device 124 detects and communicates the systolic and diastolic cycles of the heart to the controller 110. The electrocardiogram device 124 may be a stand-alone or dedicated unit operably coupled to the controller 110, or any other configuration suitable for the purposes described herein. Communication to the controller 110 may be made by any suitable communication link, for example, and not by way of limitation, a wireless connection, cable connectors, or any other means capable of allowing communication to occur between the electrocardiogram device 124 and the controller 110 of the system 100 and suitable for the purposes described herein.

The advancement motor 112, as shown in FIGS. 2-3, may be any suitable motor for providing the system 100 with the advancement/retraction of the guidewire 106 with the various functionalities described herein. The advancement motor 112 is operably coupled to the guidewire 106, as will be discussed in greater detail below. The advancement motor 112 is configured to translate or advance/retract the guidewire 106 generally in a longitudinal direction with respect to the handle assembly 102. Stated another way, the advancement motor 112 may advance and retract the guidewire 106 generally longitudinally with respect to the handle assembly 102. The advancement motor 112 is operably coupled with the controller 110 and is configured such that the controller 110 controls the direction, timing, duration (depth), and rate (speed) of the advancement motor 112, and thereby controls the advancement/retraction direction, timing, peck frequency, peck duration (depth), and peck rate (speed) of the guidewire 106 motion, disposed and coupled thereto. In the embodiment shown in FIG. 3, the advancement motor 112 is shown as comprising a linear actuator 170, a drive shaft 172, and a motor 174, disposed proximal of and coupled to the guidewire clamping device 144. The advancement motor 112 and guidewire-clamping device 144 are combined within the handle assembly 102. For instance, in an embodiment a modular motorized handle, such as a handle as disclosed in U.S. Pat. No. 9,414,916 to Costello et al., may be adapted for use in a system hereof.

However in another embodiment, the advancement motor 112 may be an external component to the handle assembly 102. Moreover, while the advancement motor 112 is shown in FIG. 3 as comprising the linear actuator 170, the drive shaft 172, and the motor 174, this is not meant to limit the design, and other configurations may be utilized, including, but not limited to roller wheel based drive assemblies, rotary motor assemblies, shaft-driven assemblies, magnetic motor assemblies, or other motor assemblies suitable for the purposes described herein.

In an embodiment, the actuator mechanism 126 is user accessible, as shown in FIG. 3. The actuator mechanism 126 is in communication with the controller 110 and is configured to selectively enable activation of the advancement motor 112 by the controller 110, thereby activating or deactivating the pecking of the guidewire 106. The actuator mechanism 126 includes an enabled (on) configuration when depressed and a disabled (off) configuration when not depressed. The actuator mechanism 126 is configured as an on/off switch such that the actuator mechanism 126 is in the disabled (off) configuration unless activated. The actuator mechanism 126 is further configured such that when the actuator mechanism 126 is in the enabled (on) configuration, the advancement motor 112 of the system 100 may be activated by the controller 110 and when the actuator mechanism 126 is in the disabled (off) configuration, the advancement motor 112 of the system 100 is not activated by the controller 110. The actuator mechanism 126 is further configured such that with the advancement motor 112 of the system 100 activated, upon transition of the actuator mechanism 126 from the enabled (on) configuration to the disabled (off) configuration, the advancement motor 112 of the system 100 deactivates. Stated another way, when the actuator mechanism 126 is depressed, the guidewire 106 of the system 100 may peck. When the actuator mechanism 126 is not depressed, the guidewire 106 of the system 100 will not peck. The actuator mechanism 126 may communicate with the controller 110 by any suitable communication link, for example, and not by way of limitation, wireless connection, cable connectors, or any other means capable of allowing communication to occur between components of the system 100 and suitable for the purposes described herein.

In another embodiment, the actuator mechanism 126 may include a mechanical input to the controller 110, an electrical, or mechanical input to the advancement motor 112, or a combination thereof to provide a user selectable on/off interface to the system 100.

While the actuator mechanism 126 is shown in FIG. 3 with the actuator mechanism 126 configured as a button, this is not meant to limit design, and other configurations may be utilized including levers, knobs, triggers, or other configurations suitable for the purposes of the system 100 described herein. Further, while the actuator mechanism 126 is shown in FIG. 3 disposed on one side of the handle assembly 102, this is not meant to limit the design, and other configurations may be utilized suitable for the purposes described herein.

The clamping device 144, as shown in FIG. 3, for clamping guidewire 106 may be any clamping device/guidewire torque/collet-gripping device known in the art. For instance in an embodiment a wire torquer available from Qosina of Ronkonkoma, N.Y. may be adapted for use in a system hereof. In an embodiment, the clamping device 144 is configured to allow the user selectable clamping, or coupling of the guidewire 106 to the advancement motor 112 such that the advancement motor 112 may advance or retract the guidewire 106, and clamping device 144, as described in greater detail below. The clamping device 144 is further configured to lock onto the guidewire 106 to prevent inadvertent longitudinal or rotational movement thereof. The clamping device 144 is disposed at a distal end of the handle assembly 102 and is configured for selectively clamping a proximal portion of the guidewire 106 to the advancement motor 112 of handle assembly 102 or selectively releasing it therefrom. Stated another way, the clamping device 144 is selectively movable from a guidewire-clamped configuration to a guidewire-released configuration. The clamping device 144 is disposed distal of and coupled to the advancement motor 112.

While the handle assembly 102 is shown in FIG. 3 with the clamping device 144 disposed distal of the advancement motor 112, this is not meant to limit the design, and other configurations may be utilized, including, but not limited to the clamping device 144 disposed proximal of the advancement motor 112, or other configurations suitable for the purposes described herein.

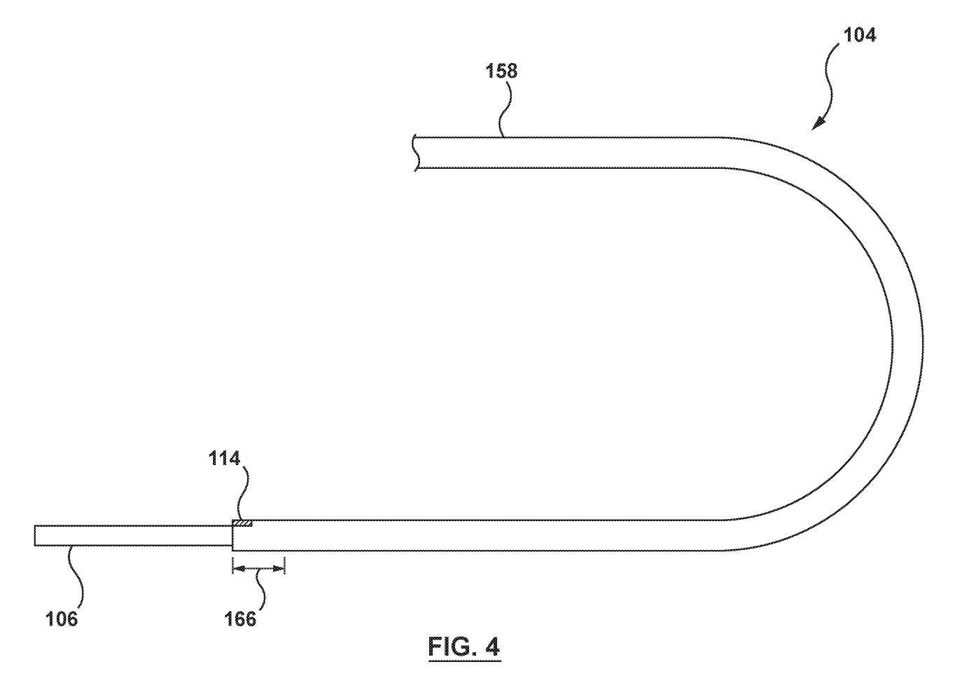

In an embodiment, the catheter 104 of the system 100 is a guide catheter. The catheter 104 includes a catheter shaft 158 and a catheter handle or hub 105, as shown in FIG. 1. The catheter 104 further includes the sensor 114 disposed at a distal portion 166 of the catheter shaft 158, as shown in FIG. 4. The catheter 104 defines a lumen that is configured to slidably receive the guidewire 106 therein. The catheter 104 is further configured to provide communication with the controller 110 (not shown in FIG. 4) from the sensor 114 as described in greater detail below. Catheter 104 is an elongate tubular structure that may be formed from any suitable materials, such as, but not limited to polyethylene (PE), polyethylene terephthalate (PET), polyvinylchloride PVC), polyether block amide (PEBAX), Nylon 12, or any other materials suitable for the purposes described herein.

The sensor 114 of the catheter 104, as shown in FIG. 4, may be any sensor suitable for sensing a condition at a distal portion of the guidewire 106 and communicating with the controller 110. In an embodiment, the sensor 114 is a force or pressure sensor, suitable for providing the system 100 with the various functionalities described herein. In an embodiment, the sensor 114 is configured to generate and communicate a continuous signal of the resistance force (pressure) on the tip 169 of the guidewire 106 to the controller 110. For instance, in an embodiment a force-sensor may be adapted for use in a system hereof. In an embodiment shown in FIG. 4, the sensor 114 is disposed at and coupled to the distal portion 166 of the catheter shaft 158. The sensor 114 is in communication with the controller 110, as previously described. The sensor 114 is coupled to the distal portion 166 of the catheter shaft 158 in a manner such as, but not limited to adhesives, or other methods suitable for the purposes disclosed herein.

The guidewire 106 may be any suitable guidewire, as is known in the art, for crossing a native heart valve. The guidewire 106 is an elongate structure that includes a proximal end (not shown) that extends proximal of the handle assembly 102 and the tip 169, also known as a distal end, as shown in FIG. 1. The guidewire 106 is configured to traverse the vasculature of a patient and cross the native heart valve of a beating heart at a treatment site. The guidewire 106, when not held by clamping device 144, is moveably disposed within the handle assembly 102 thereof.

With an understanding of the components of the system 100 above, it is now possible to describe the interactions of the various components and inputs of the system 100. As previously described, the system 100 of FIGS. 1-4 includes the guidewire 106 having the handle assembly 102, and the catheter 104. The guidewire 106 is configured to cross the native heart valve of a beating heart. Information from the electrocardiogram device 124 is communicated to the controller 110 of the system 100. The controller 110 determines the systolic phase and the diastolic phase of the patient's heart from analyzing a plurality of cycles (a non-limiting example being the previous 10 cycles) of the patient's heart, received from the electrocardiogram device 124. Utilizing the systolic phase and diastolic phase determination, the controller 110 calculates when the native heart valve to be crossed is in the open configuration. With the above determination and completed calculation, and with the actuator mechanism 126 in the enabled (on) configuration, the system 100 begins advancing (pecking) the guidewire 106 to coincide with the next open configuration of the native heart valve. The controller 110 advances the guidewire 110 by activating the advancement motor 112. The controller 110 controls the advancement (peck) of the guidewire 106 and times the advancement (peck) to coincide with the open configuration of the native heart valve. The controller 110 further controls the guidewire advancement (peck) speed (mm/sec) from the user selectable peck speed adjustment 116, the guidewire advancement (peck) frequency (pecks per minute (ppm)) from the user selectable peck frequency adjustment 118, the depth of guidewire advancement (peck) (longitudinal distance (mm)) from the user selectable peck depth adjustment 120, and the timing of the guidewire advancement (peck) (advancement/delay before/after systolic peak (0-1000 msec)) from the user selectable systolic/diastolic delay adjustment 122.

In an embodiment, the clinician monitors the guidewire 106 via fluoroscopy or any other method suitable for the purposes described herein. Upon determination of an unsuccessful crossing, the clinician releases actuator mechanism 126 to the disabled (off) configuration, stopping the pecking cycle. The clinician may manipulate the position of the guidewire 106. The crossing may be reattempted by implementing a new peck cycle as previously described. The repositioning of the guidewire 106 and the reimplementation of a new pecking cycle may be repeated until the guidewire 106 successfully crosses the native heart valve as determined by the clinician.

In another embodiment, the sensor 114 continuously monitors and communicates the resistance force (pressure) on the tip 169 of the guidewire 106 with the controller 110. Upon determination by the controller 110 of the resistance force (pressure) on the tip 169 of the guidewire 106 that is indicative of the tip 169 of the guidewire 106 advancing into tissue (i.e. not crossing the native heart valve), the controller 110 will stop advancement of the guidewire 106. The actuator mechanism 126 is released to the disabled (off) configuration, and the clinician may manipulate the position of the guidewire 106. The crossing may be reattempted by implementing a new peck cycle as previously described. The repositioning of the guidewire 106 and the reimplementation of a new pecking cycle may be repeated until the guidewire 106 successfully crosses the native heart valve as determined by the clinician.

In another embodiment, the sensor 114 continuously monitors and communicates the resistance force (pressure) on the tip 169 of the guidewire 106 with the controller 110. Upon determination by the controller 110 of the resistance force (pressure) on the tip 169 of the guidewire 106 that is indicative of the tip 169 of the guidewire 106 advancing into tissue (i.e. not crossing the native heart valve), the controller 110 will retract the guidewire 106 a preset distance such that the guidewire 106 does not damage the native heart valve or surrounding tissue. The actuator mechanism 126 is released to the disabled (off) configuration, and the clinician may manipulate the position of the guidewire 106. The crossing may be reattempted by implementing a new peck cycle as previously described. The repositioning of the guidewire 106 and the reimplementation of a new pecking cycle may be repeated until the guidewire 106 successfully crosses the native heart valve as determined by the clinician.

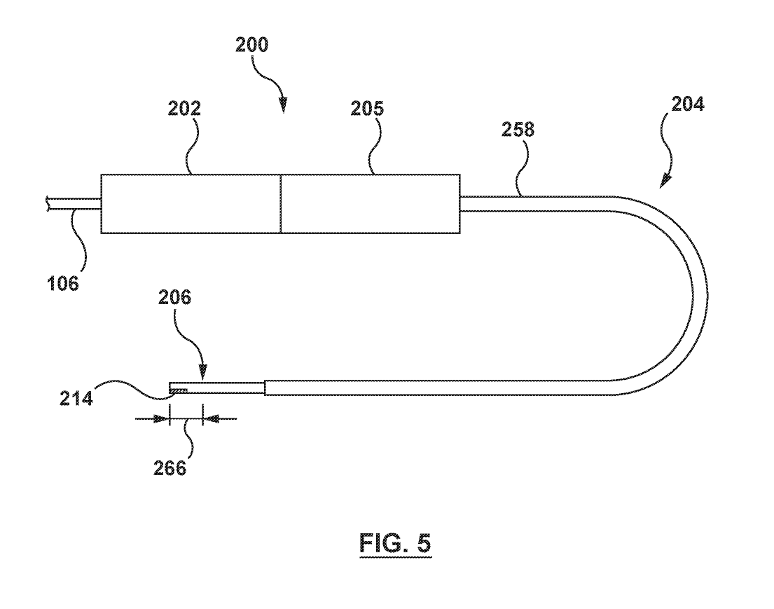

FIG. 5 illustrates a system 200 for driving or pecking a guidewire based on an electrocardiogram in accordance with another embodiment hereof. Similar to the system 100, the system 200 for crossing a native heart valve of a beating heart includes a guidewire 206 having a handle assembly 202, and a catheter 204. The handle assembly 202 is similar to handle assembly 102, the catheter 204 is similar to the catheter 104, and the guidewire 206 is similar to the guidewire 106. Therefore, details of the handle assembly 202, the catheter 204, and the guidewire 206, are not repeated here.

In an embodiment, the catheter 204 includes a catheter shaft 258 and a handle or hub 205. The handle 205 of the catheter 204 is coupled to the handle assembly 202. The catheter shaft 258 is similar to the catheter shaft 158 (FIG. 1), and the handle 205 is similar to the catheter handle 105 (FIG. 1). Therefore, details of the catheter shaft 258 and the handle 205 are not repeated here. The handle 205 may be coupled to the handle assembly 202 in a manner such as, but not limited to a mechanical coupling, adhesives, or other methods suitable for the purposes disclosed herein.

Guidewire 206 may be any guidewire, as is known in the art, for crossing a native heart valve of a beating heart and suitable for providing the system 200 with the various functionalities described herein. Guidewire 206 further includes a sensor 214 disposed at a distal portion 266 of guidewire 206, as shown in FIG. 5. Guidewire 206 is configured to traverse the vasculature of a patient and cross the native heart valve at a treatment site.

Sensor 214 of guidewire 206, as shown in FIG. 5, is similar to the sensor 114 (FIG. 4). Therefore, the details of the sensor 214 are not repeated here. However, unlike the sensor 114, the sensor 214 is disposed at and coupled to the distal portion 266 of the guidewire 206. The sensor 214 may be coupled to the distal portion 266 of the guidewire 206 in a manner such as, but not limited to, adhesives, or other methods suitable for the purposes disclosed herein.

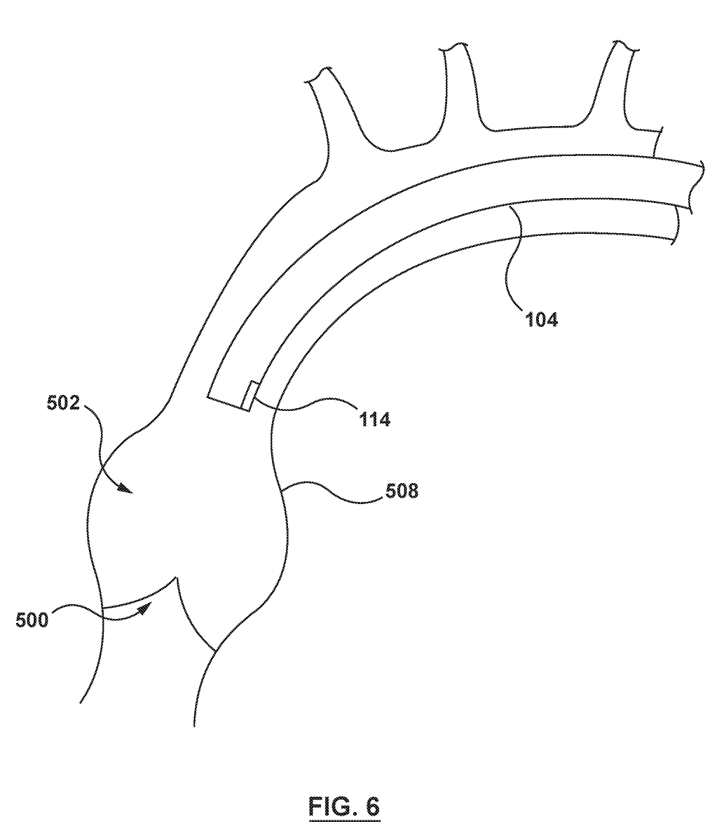

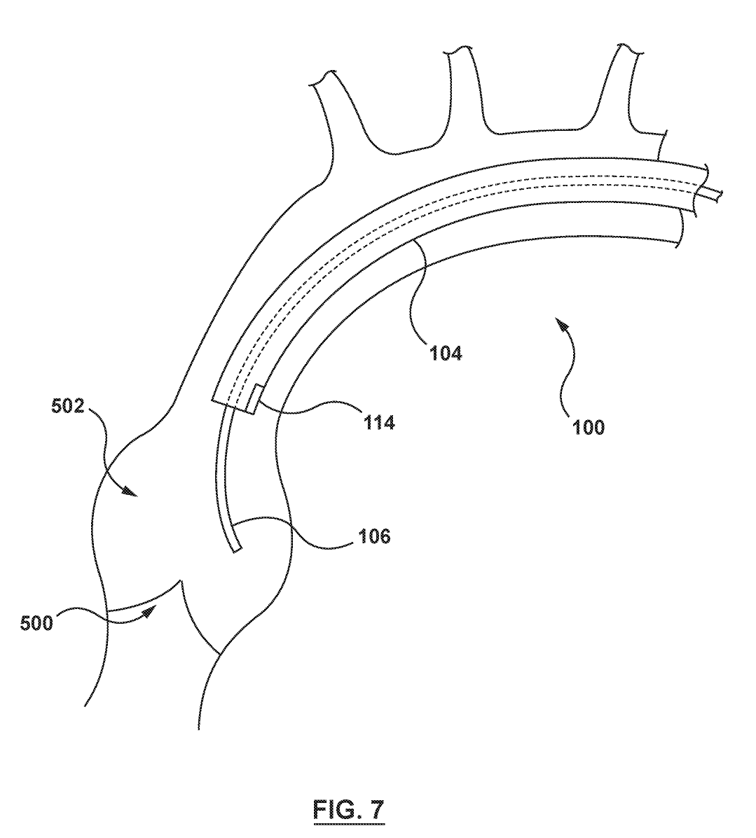

FIGS. 6-15 illustrate schematically an embodiment of a method of crossing a native heart valve of a beating heart with a system 100. Using established percutaneous transcatheter procedures, a catheter 104 of the system 100 is introduced into a patient's vasculature and positioned at a desired treatment site distal or downstream of an aortic sinus 508, as shown in FIG. 6. A guidewire 106 is introduced into the catheter 104 and advanced therethrough into the patient's vasculature using established percutaneous transcatheter procedures to the desired treatment site. The guidewire 106 is positioned at a first side 502 of an aortic valve 500, as shown in FIG. 7. Although the method is described herein using the system 100, it will be apparent to one of ordinary skill that the methods described herein may utilize a system according to any embodiment described herein. The guidewire 106 and the catheter 104 of system 100 may also include, for example, radiopaque markers such that clinician may determine when guidewire 106 is in the proper location for advancement (pecking) and crossing of aortic valve 500.

With the guidewire 106 at the desired location, the electrocardiogram device 124 (not shown in FIGS. 6-15) of the system 100 detects a plurality of cycles of the patient's heart and communicates the information/data to the controller 110 (not shown in FIGS. 6-15). The controller 110 determines the patient's systolic phase from the information/data received from the electrocardiogram device 124 that corresponds with the aortic valve 500 being in an open configuration. Further, the controller 110 calculates an interval for repetitive automatic advancement (pecking) of the guidewire 106 from the information/data received from the electrocardiogram device 124.

The clinician manipulates the actuator mechanism 126 (not shown in FIGS. 6-15) and the actuator mechanism 126 (not shown in FIGS. 6-15) transitions from the disabled (off) configuration to the enabled (on) configuration. When the aortic valve 500 is in the open configuration as determined by the controller 110 and the actuator mechanism 126 is in the enabled (on) configuration, the controller 110 activates the advancement motor 112 (not shown in FIGS. 6-15) of the system 100 and the advancement motor 112 advances (pecks) the guidewire 106 in a direction D1 towards a second side 506 of the aortic valve 500, as shown in FIG. 8.

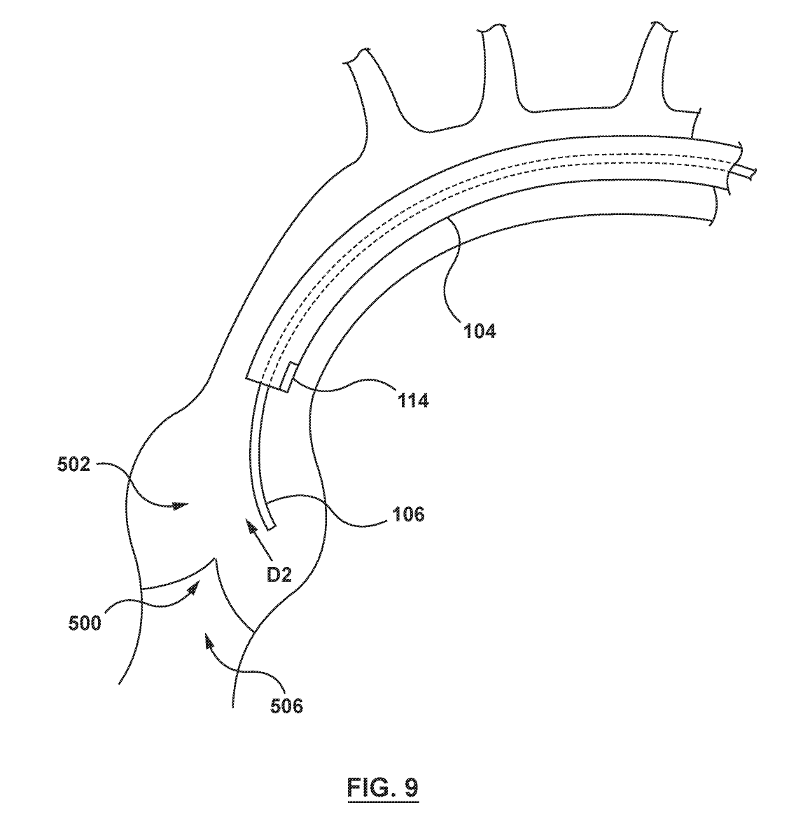

If the guidewire 106 does not cross from the first side 502 to the second side 506 of the aortic valve 500 as determined by the clinician using fluoroscopy or other methods known to the art, or if a resistance force (pressure) on the tip 169 of the guidewire 106 sensed by the sensor 114 and communicated with the controller 110 is determined by the controller 110 to be indicative of the guidewire not advancing between open leaflets of the aortic valve, the guidewire 106 is retracted in a direction D2, wherein the direction D2 is opposite the direction D1, as shown in FIG. 9.

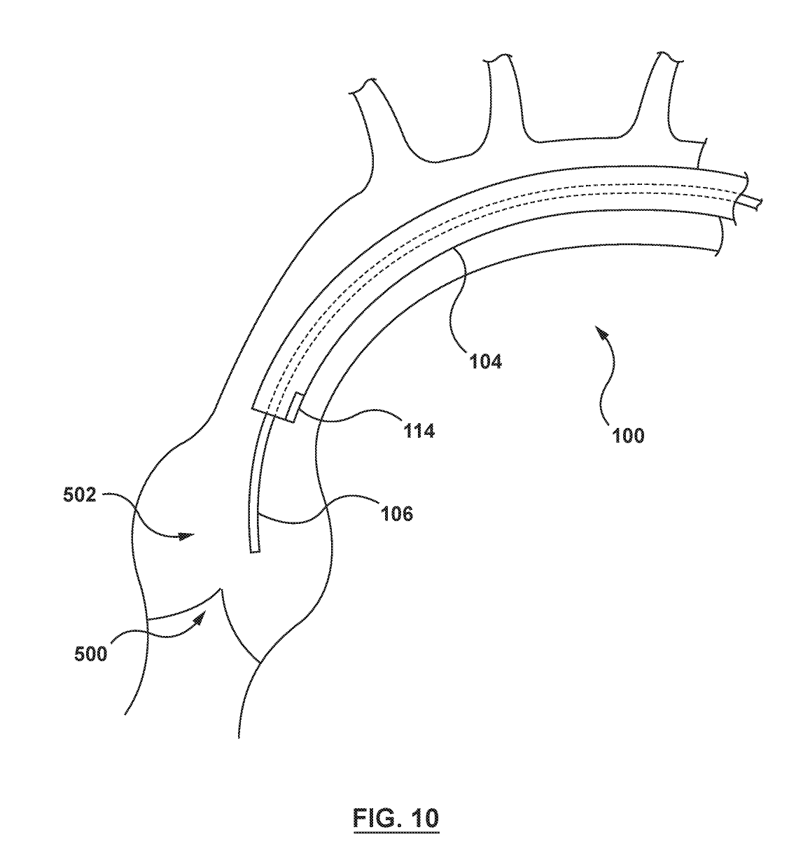

The clinician may rotate or otherwise maneuver the system 100 such that the guidewire 106 is repositioned at a different location on the first side 502 of the aortic valve 500, as shown in FIG. 10.

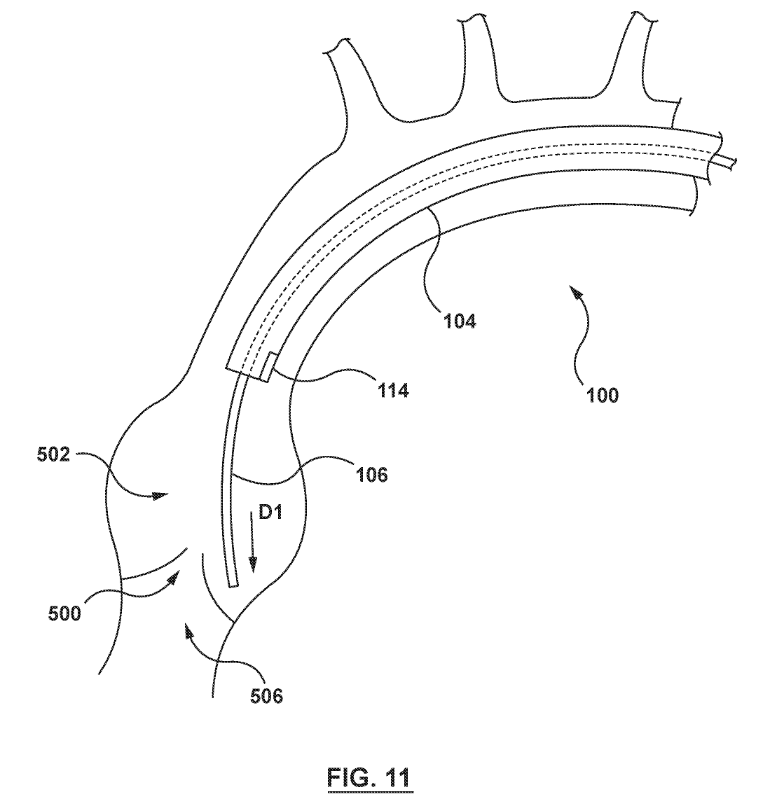

When the aortic valve 500 is in the open configuration as determined by the controller 110 and the actuator mechanism 126 is in the enabled (on) configuration, the controller 110 again activates the advancement motor 112 of the system 100 and the advancement motor 112 advances (pecks) the guidewire 106 in the direction D1 towards the second side 506 of the aortic valve 500, as shown in FIG. 11.

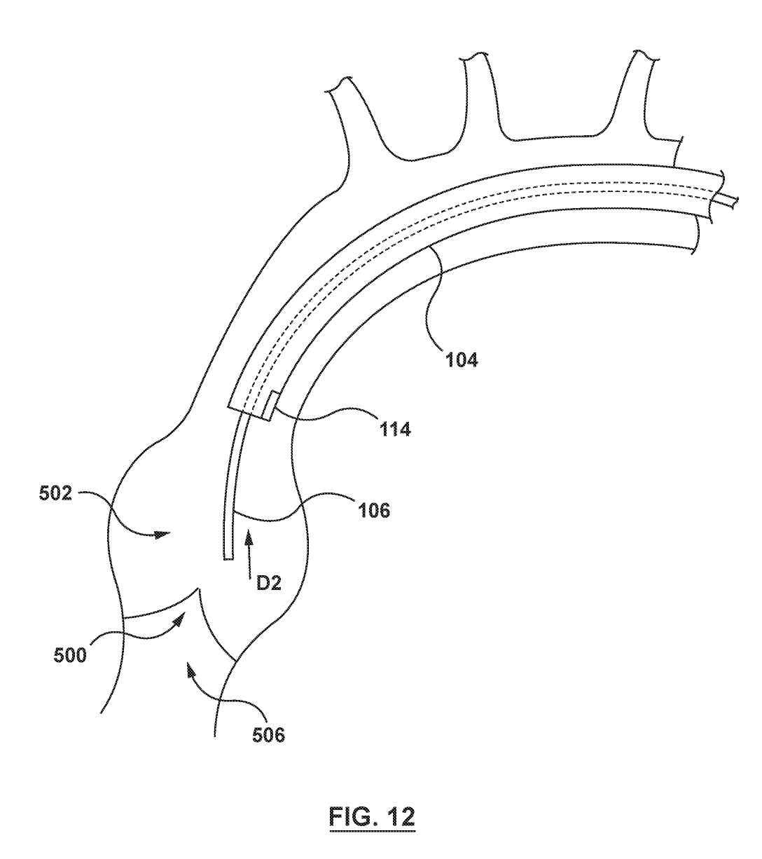

If the guidewire 106 does not cross from the first side 502 to the second side 506 of the aortic valve 500 as determined by the clinician using fluoroscopy or other methods known to the art, or if the resistance force (pressure) on the tip 169 of the guidewire 106 sensed by the sensor 114 and communicated with the controller 110 is determined by the controller 110 to be indicative of the guidewire not advancing between open leaflets of the aortic valve, the guidewire 106 is automatically retracted in the direction D2, wherein the direction D2 is opposite the direction D1, as shown in FIG. 12.

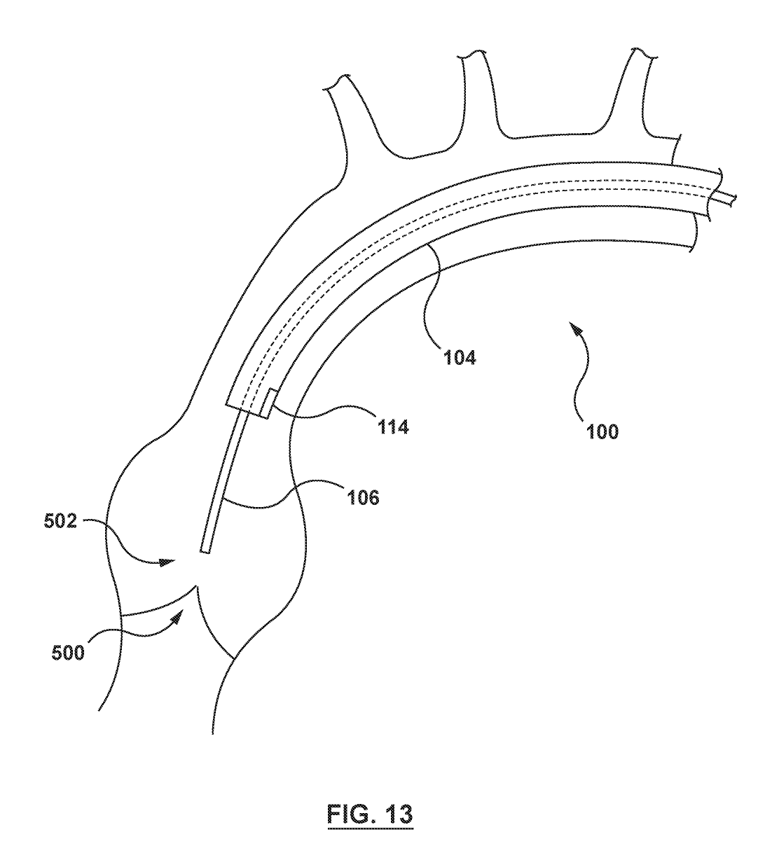

The clinician once again may rotate or otherwise maneuver the system 100 such that the guidewire 106 is repositioned at a different location on the first side 502 of the aortic valve 500, as shown in FIG. 13.

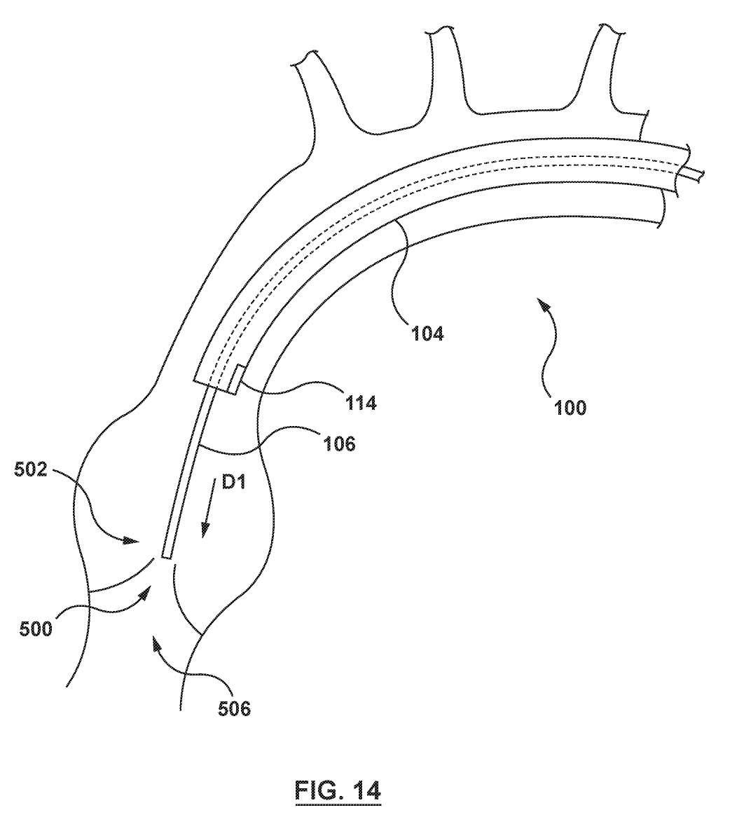

When the aortic valve 500 is in the open configuration as determined by the controller 110 and the actuator mechanism 126 is in the enabled (on) configuration, the controller 110 once again activates the advancement motor 112 of the system 100 and the guidewire 106 advances (pecks) in the direction D1 towards the second side 506 of the aortic valve 500, as shown in FIG. 14.

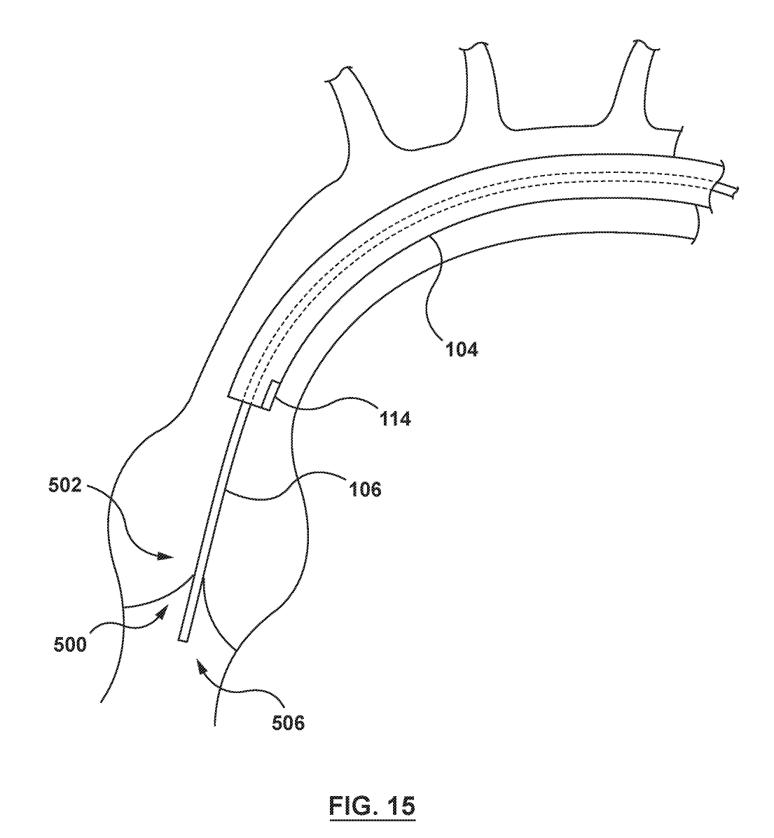

When the clinician determines the guidewire 106 has advanced from the first side 502 to the second side 506 of the aortic valve 500, as shown in FIG. 15, the actuator mechanism 126 is manipulated by the clinician, such that the actuator mechanism 126 transitions from the enabled (on) configuration to the disabled (off) configuration, and pecking of the guidewire 106 ceases.

While the method of FIGS. 6-15 describes adjusting the location of the guidewire 106 two (2) times and advancing the guidewire 106 three (3) times to cross the aortic valve 500, it is for illustrative purposes only and more or fewer location adjustments, advancements, and retractions may be required for the guidewire 106 to cross from the first side 502 to the second side 506 of the aortic valve 500.

Additionally, while the method of FIGS. 6-15 describes the actuator mechanism 126 as remaining in the engaged configuration for the duration of the method described above, this is not meant to limit uses hereof, and the actuator mechanism 126 may be manipulated at any time at the discretion of the clinician.

While the method of FIGS. 6-15 illustrates an embodiment of the system 100 with the aortic valve 500, those skilled in the art will understand that the method described with FIGS. 6-15 would also apply to other native valve locations including, but not limited to a mitral valve, a pulmonary valve, and a tricuspid valve.

While the method of FIGS. 6-15 illustrates an embodiment of the system 100 with the catheter 104 and the sensor 114, those skilled in the art will understand that the method described with FIGS. 6-15 would also apply to other embodiments without the catheter 104 and/or the sensor 114.

Although the method of FIGS. 6-15 are described above using the embodiment of the system 100 of FIGS. 1-4, the method is applicable with other embodiments of the invention including the system 200 of FIG. 5. It has been described herein with the embodiment of the system 100 of FIGS. 1-4 for convenience.

While only some embodiments have been described herein, it should be understood that it has been presented by way of illustration and example only, and not limitation. Various changes in form and detail can be made therein without departing from the spirit and scope of the invention, and each feature of the embodiments discussed herein, and of each reference cited herein, can be used in combination with the features of any other embodiment. All patents and publications discussed herein are incorporated by reference herein in their entirety.

What is claimed is:

1. A system for crossing a heart valve of a beating heart with a guidewire, the system comprising:
an advancement motor, wherein the advancement motor is configured to be connected to the guidewire and is configured to advance and retract the guidewire; and
a controller coupled to the advancement motor, wherein the controller is configured to control when the advancement motor advances and retracts the guidewire, wherein the controller is configured to be coupled to an electrocardiogram device, and wherein the controller is configured to determine the systolic phase and the diastolic phase of a patient's heart from information/data received from the electrocardiogram device and wherein the advancement motor is configured to advance the guidewire based on the controller's determination of the systolic phase or the diastolic phase corresponding with the heart valve being in an open configuration.

2. The system of claim 1, wherein the controller is configured to determine the systolic phase and the diastolic phase of the patient's heart by analyzing a plurality of systolic and diastolic cycles of the heart and predicting the systolic and diastolic phases.

3. The system of claim 1, further comprising:
a catheter including a lumen through which the guidewire is disposed.

4. The system of claim 1, further comprising:
a sensor in communication with the controller, wherein the controller is configured to stop advancement of the guidewire if the controller determines the guidewire has not advanced between open leaflets of the heart valve based upon information/data from the sensor.

5. The system of claim 4, wherein the sensor is coupled to a distal portion of a catheter within which the guidewire is slidably disposed.

6. The system of claim 4, wherein the sensor is coupled to a distal portion of the guidewire.

7. The system of claim 4, wherein the controller is configured to retract the guidewire when the sensor senses a resistance force that is indicative of or corresponds to the guidewire advancing into tissue rather than between open leaflets of the heart valve.

8. The system of claim 4, wherein the sensor is a force sensor, wherein the force sensor is configured to sense a resistance force that is indicative of or corresponds to the guidewire advancing into tissue rather than between open leaflets of the heart valve.

9. A method for crossing a native heart valve of a beating heart with a guidewire, the method comprising the steps of:
advancing the guidewire to a first side of the native heart valve, wherein the guidewire is coupled to an advancement motor configured to advance and to retract the guidewire;
monitoring the patient's heart with an electrocardiogram device to detect systolic and diastolic phases of the heart, wherein the electrocardiogram device is coupled to a controller which is coupled to the advancement motor;
advancing the guidewire from the first side of the native heart valve towards a second side of the native heart valve automatically using the advancement motor when the controller determines from information/data received from the electrocardiogram device that the heart is in the systolic phase or the diastolic phase that corresponds with the heart valve being in an open configuration; and
retracting the guidewire if the guidewire does not advance to the second side of the native heart valve.

10. The method of claim 9, further comprising the steps of:
adjusting the location of the guidewire after the retracting step; and
repeating the advancing, retracting, and adjusting steps until the guidewire advances through the open native heart valve to the second side of the native heart valve.

11. The method of claim 10, wherein the step of advancing the guidewire to the first side of the native heart valve further includes advancing a catheter with a lumen through which the guidewire is disposed to the first side of the native heart valve.

12. The method of claim 10, wherein the step of adjusting the location the guidewire comprises a user manipulating or maneuvering the catheter.

13. The method of claim 9, wherein the step of monitoring the patient's heart further comprises the controller calculating an interval for repetitive automatic advancement of the guidewire based on analyzing a plurality of cycles of the patient's heart and calculating when the heart valve will be in the open configuration.

14. The method of claim 9, further comprising the steps of:
sensing a condition at a distal portion of a guidewire utilizing a sensor in communication with the controller; and
automatically stopping the advancement of the guidewire utilizing the controller if the sensor senses a condition indicating that the guidewire has not advanced between open leaflets of the native heart valve.

15. The method of claim 14, wherein in the sensor is coupled to a distal portion of the catheter.

16. The method of claim 14, wherein the sensor is coupled to a distal portion of the guidewire

17. The method of claim 14, wherein the sensor is a force sensor, wherein the force sensor is configured to sense a resistance force that is indicative of or corresponds to the guidewire advancing into tissue rather than between open leaflets of the heart valve.

18. The method of claim 14, further comprising the step of:
automatically retracting the guidewire when the sensor senses a condition indicating that the guidewire has not advanced between open leaflets of the native heart valve.

19. The method of claim 9, wherein the heart valve is an aortic valve, the first side of the heart valve is a downstream side of the aortic valve, and the advancing step occurs in the systolic phase.

20. The method of claim 9, wherein the heart valve is a mitral valve and the advancing step occurs in the diastolic phase.
